(19) United States
(12) Patent Application Publication
(10) Pub. No.: US 2021/0308078 A1
(43) Pub. Date: Oct. 7, 2021

(54) NEW USE OF INHIBITORS OF MONOAMINE OXIDASE TYPE B

(71) Applicant: ISTITUTO Di RICERCA PEDiATRiCA CITTA DELLA SPERANZA, Padova (IT)

(72) Inventors: Antonella VIOLA, Padova (IT); Marcella CANTON, Padova (IT)

(21) Appl. No.: 17/253,940

(22) PCT Filed: Jun. 18, 2019
(86) PCT No.: PCT/IB2019/055068

§ 371 Date: Dec. 18, 2020

(30) Foreign Application Priority Data
Jun. 19, 2018 (IT) 102018000006455

(51) Int. Cl. A61K 31/165; A61K 31/137; A61K 9/00

(52) U.S. Cl. CPC A61K 31/165; A61K 9/0019; A61K 31/137

(57) ABSTRACT

The invention relates to inhibitors of monoamine oxidase type B (iMAO-B) for use in the treatment of pathologies or disorders associated with inflammasome activation, pharmaceutical compositions comprising said inhibitors for use thereof, medical devices comprising such compositions, and therapeutic methods for treatment of pathologies or disorders associated with inflammasome activation by administration of inhibitors of the monoamine oxidase type B enzyme.

The invention relates to inhibitors of monoamine oxidase type B (iMAO-B) for use in the treatment of pathologies or disorders associated with inflammasome activation, pharmaceutical compositions comprising said inhibitors for use thereof, medical devices comprising such compositions, and therapeutic methods for treatment of pathologies or disorders associated with inflammasome activation by administration of inhibitors of the monoamine oxidase type B enzyme.

### PRIOR ART

It is known in the literature that inflammasome activation is associated with numerous pathologies. Consequently, many studies have focussed on finding new therapies aimed at inhibiting the inflammasome. Regarding autoinflammatory diseases (i.e. CAPS), biotechnological drugs are used that act by reducing the detrimental effects of the excessive release of IL-1β and IL-18 caused by inflammasome hyperactivation. However, these molecules, which include the IL-1 receptor antagonist (anakinra) or the neutralising antibody for IL-1β, are not able to inhibit caspase-1 activation. This constitutes a limitation because caspase-1 cleaves other proteins in addition to pro-IL-1β and pro-IL-18, (such as gasdermin D), this contributing for example to pyroptosis, which is a type of cell death which releases DAMPs, which intensify the pathological condition. In addition, the costs to obtain these drugs (recombinant proteins) are still extremely high.

Inflammation is a protective immune response, triggered by the innate immune system, which was preserved during evolution, in response to harmful stimuli, such as pathogens, cell death or irritating substances, and is finely regulated by the host. A weak inflammatory response can lead to persistent pathogenic infections, whereas excessive activation of the immune response can lead to chronic diseases or systemic inflammatory diseases, such as sepsis.

Among the diseases associated with the inflammasome, one of those having the most harmful consequences is sepsis.

Sepsis is the generalised inflammatory response of an organism to an attack by a pathogenic agent. It is one of the main causes of death and morbidity in adult patients hospitalised in intensive care and in patients of paediatric age in industrialised countries. Currently, the fundamental principle for reducing death and morbidity induced by sepsis is based on two mechanisms: the early start of effective antibiotic treatment and reduction of systemic inflammatory response syndrome (SIRS), which causes multi-organ dysfunction and death. In the last decade, a rising number of studies has shown that the activation of the inflammasomes plays a key role in this pathology.

Many studies have shown the importance of the involvement of the inflammasomes in the pathogenesis of numerous pathologies (Ayres, J. S., Trinidad, N. J., and Vance, R. E. (2012). Lethal inflammasome activation by a multidrug-resistant pathobiont upon antibiotic disruption of the microbiota. Nature Med 18, 799-806; Kalbitz, M., Fattahi, F., Grailer, J. J., Jajou, L., Malan, E. A., Zetoune, F. S., Huber-Lang, M., Russell, M. W., and Ward, P. A. (2016). Complement-induced activation of the cardiac NLRP3 inflammasome in sepsis. FASEB journal: official publication of the Federation of American Societies for Experimental Biology 30, 3997-4006; Hao, H., Cao, L., Jiang, C., Che, Y., Zhang, S., Takahashi, S., Wang, G., and Gonzalez, F. J. (2017). Farnesoid X Receptor Regulation of the NLRP3 Inflammasome Underlies Cholestasis-Associated Sepsis. Cell Metab 25, 856-867 e 855; Pu, Q., Gan, C., Li, R., Li, Y., Tan, S., Li, X., Wei, Y., Lan, L., Deng, X., Liang, H., et al. (2017). Atg7 Deficiency Intensifies Inflammasome Activation and Pyroptosis in Pseudomonas Sepsis. The Journal of Immunology).

Their clinical relevance goes beyond infectious diseases, since an absence of their regulation is correlated with various inflammatory pathologies, both congenital and acquired. In fact, strict control of the assembly and signalling of the inflammasomes is crucial, because the immune system must be allowed to activate the antimicrobial and inflammatory response in order to block the pathogenic infection, but at the same time excessive activation must be avoided, since this causes damage to the tissues and consequently leads to chronic or systemic inflammatory diseases. Within the family of the inflammasomes, NLRP3 is the one that is best characterised. In the activation of the inflammasome NLRP3, the initial input is provided by extracellular inflammatory stimuli (for example lipopolysaccharides, LPS), which induce an over-expression of the protein NLRP3 and of pro-inflammatory cytokines, followed by a series of stimuli, such as extracellular ATP, ionophore toxins (such as nigericin) or crystals (for example uric acid), which promote the assembly of said inflammasome. The variety of stimuli suggests that a common cellular event is at the root of the activation. However, although numerous studies have been conducted over the years, there is still no unifying molecular mechanism, as summarised in Broz, P., and Dixit, V. M. (2016). Inflammasomes: mechanism of assembly, regulation and signalling. Nature reviews Immunology 16, 407-420. The various mechanisms that have been proposed include: potassium efflux, lysososomal destabilisation, cytosolic mitochondrial DNA release, mitochondrial dysfunction, and subsequent production of reactive oxygen species (ROS). Although it is commonly accepted that the mitochondria are the primary site of ROS production, the molecular sources responsible for the activation of NLRP3mediated by ROS are still themselves unknown. The ROS can be produced by means of various mechanisms, such as electron loss (leak) at the level of respiratory chain complexes, or as by-products of the activity of various oxidases (such as NADPH oxidases or monoamine oxidases). The mitochondria are also the primary target of ROS, due to the large quantity of thiols present. In fact, in pathological conditions, the excess of ROS attacks the mitochondria, causing their malfunction, which then leads to a further production of ROS in a never-ending cycle. It is interesting to cite a recent study underlining the crucial role of mitochondrial ROS in the production of pro-inflammatory cytokines induced by LPS and in the excessive reactivity to LPS in the autoinflammatory disease called TRAPS (Bulua, A. C., Simon, A., Maddipati, R., Pelletier, M., Park, H., Kim, K. Y., Sack, M. N., Kastner, D. L., and Siegel, R. M. (2011). Mitochondrial reactive oxygen species promote production of proinflammatory cytokines and are elevated in TNFR1-associated periodic syndrome (TRAPS). The Journal of experimental medicine 208, 519-533). This study also demonstrated that the ROS produced by NADPH oxidases do not have any pro-inflammatory action. In spite of the increasing interest in, and the growing number of studies focussed on the characterisation of both the molecular sources for production of mitochondrial ROS and the relationship between mitochondrial dysfunction and inflammasome activation in pathological conditions, numerous questions remain unanswered.

The identification of molecules able to inhibit inflammasome activation might offer better therapeutic prospects, and in fact various molecules are currently being studied. For example, a comprehensive characterisation has been performed for the pharmacokinetics in vitro and in vivo of a molecule named MCC950, which constitutes a significant step towards therapeutic application, in spite of the still unclear mechanism of inhibition of NLRP3. The finding that the ketone body β-hydroxybutyrate, which is produced physiologically by the liver during fasting as an alternative source of ATP, inhibits activation of the inflammasome NLRP3, in spite of the fact that the direct target of this molecule has not yet been defined, is of great interest.

### SUMMARY OF THE INVENTION

The authors of the present invention have surprisingly found a link between the mitochondrial monoamine oxidase type B enzyme (MAOB) and inflammasome activation, which is, as explained above, a complex multiprotein which induces the maturation and secretion of pro-inflammatory cytokines. Both protein levels and the MAOB-dependent production of hydrogen peroxide are increased when the inflammasome in isolated murine and human macrophages and activated. The inhibition of the enzyme by way of drugs for clinical use significantly reduces ROS production and mitochondrial dysfunction, in parallel with a decrease in inflammasome activity and subsequent release of pro-inflammatory cytokines. The inhibition of MAOB decreases the expression of proteins that are essential for inflammasome activity, i.e. interleukin 1 beta and NLRP3.

The studies performed by the inventors indicate that the production of ROS by means of MAOB induces mitochondrial dysfunction (depolarisation), inflammasome activation, and subsequently pro-inflammatory cytokine release. This is consistent with previous data, placing mitochondrial ROS in relation with the activation of such multiprotein complex, and the idea that the accumulation of damaged (depolarised) mitochondria is a common denominator in the activation of the inflammasome NLRP3. Such studies, however, although they are of considerable interest insofar as they underline the importance of mitochondrial ROS in inflammation, they do not identify a specific ROS source.

The studies performed by the inventors, shown in the drawings and discussed further below in the present description, identify the most significant ROS source for inflammasome activation as being the mitochondrial monoamine oxidase enzyme from a translational viewpoint, this observation is of significant interest insofar as the structure of the enzyme was characterised under X-ray and many inhibitors, currently already in use in clinical practice, were named.

MAO inhibitors (iMAO) are drugs of interest insofar as they prevent the formation of a specific fraction of mitochondrial ROS which become excessive in pathological conditions, in contrast to generic antioxidants, which remove aspecifically the ROS that have already formed. In this regard it should be noted that the controlled production of ROS is physiologically necessary for intracellular signalling, and it has been hypothesised that this is one of the reasons for the weak efficacy of antioxidant therapy by means of generic ROS scavengers. In addition, in contrast to the inhibitors of monoamine oxidase type A, iMAO-B have the advantage of not causing the severe side-effects which are characteristic of the former, such as hypertensive crisis (what is known as the cheese effect, caused by the ingestion of foods rich in tyramine).

A further advantage is constituted by the fact that the translation to clinical application should be facilitated insofar as these drugs have already been approved for neurological pathologies. For example, the inhibitor of MAOB rasagiline is a well-tolerated molecule, the use of which was approved in Europe and in the United States in 2005/2006 for the treatment of Parkinson's disease.

The following thus form the subject of the invention:

an inhibitor of the monoamine oxidase type B enzyme, iMAO-B, for use in the treatment of pathologies or disorders associated with inflammasome activation or, in other words, presenting inflammasome activation;

a pharmaceutical composition comprising one or more inhibitors of monoamine oxidase type B (iMAO) and at least one pharmaceutically acceptable carrier for use in the treatment of pathologies or disorders associated with inflammasome activation;

a medical device comprising a pharmaceutical composition comprising one or more inhibitors of monoamine oxidase type B (iMAO-B) and at least one pharmaceutically acceptable carrier, and a therapeutic method for the treatment of pathologies or disorders associated with inflammasome activation, in which an inhibitor of the monoamine oxidase type B enzyme (iMAO-B) is administered in therapeutically effective doses or a pharmaceutical composition comprising one or more inhibitors of monoamine oxidase type B (iMAO-B) and at least one pharmaceutically acceptable carrier is administered. Subsequently, the patient can be treated by the above-described therapy and/or with use of a medical device comprising a pharmaceutical composition comprising one or more inhibitors of monoamine oxidase type B.

### DETAILED DESCRIPTION OF THE DRAWINGS

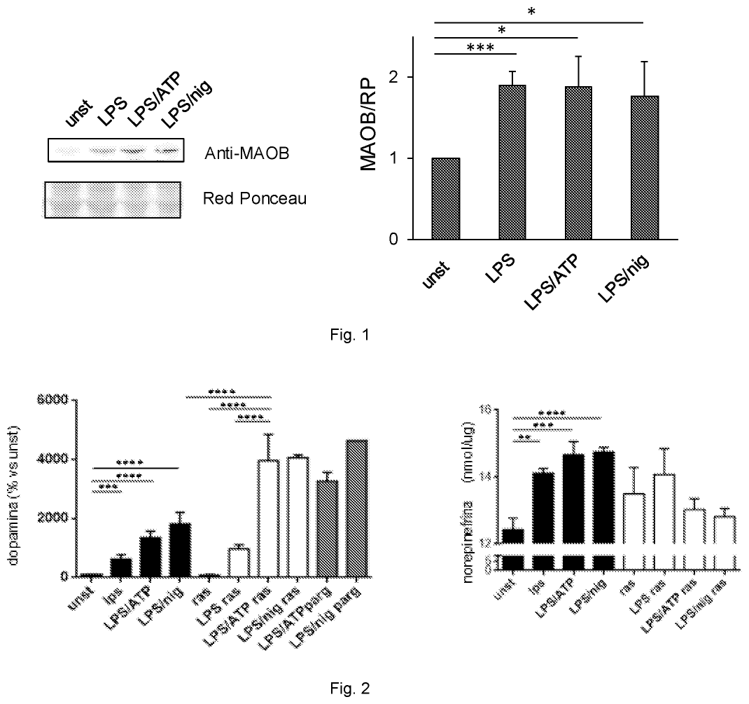

FIG. 1. The protein levels of MAO-B rise in response to inflammatory stimuli. Human monocytes isolated from the buffy coat were differentiated with GM-CSF (10 ng/ml) for 7 days and then stimulated for 8 h with only LPS (100 ng/ml), LPS (100 ng/ml)+ATP (1 mM), LPS (100 ng/ml)+nigericin (nig, 15 μM, added the last 30 min) or were unstimulated (unst). The total lysates were separated by means of SDS/PAGE and transferred to nitrocellulose membrane. The figure shows an example of Western blot with anti-MAOB antibodies (box on the left) and the relative quantification of the intensity of the bands by means of densitometric analysis (box on the right). The control of the correct loading of the proteins was performed by means of membrane staining with Ponceau S (RP). The intensity of the band of MAOB was normalised by the amount of loaded proteins. The values are the average of at least 3 independent experiments±SEM. The figure shows that the pro-inflammatory treatments induce a significant rise in the protein levels of the enzyme.

FIG. 2. The substrates of MAO rise in response to inflammatory stimuli. Human monocytes isolated from the buffy coat were differentiated with GM-CSF (10 ng/ml) for 7 days and then stimulated for 8 h as described in FIG. 1, in the absence or presence of pargyline (parg, 100 μM) or rasagiline (ras, 5 μM). The intracellular levels of dopamine and norepinephrine (both substrates of MAO) were measured by means of mass spectrometry on cell lysates extracted with methanol:water (70:30). Such levels increase significantly in the presence of LPS, LPS/ATP and LPS/nig. The concentration of dopamine, in contrast to that of norepinephrine, increases subsequently when its consumption is inhibited, thus blocking the MAO with both the inhibitors. This result indicates that the increase in intracellular production of dopamine is used by the MAO enzyme. The values are the average of at least 3 independent experiments±SEM.

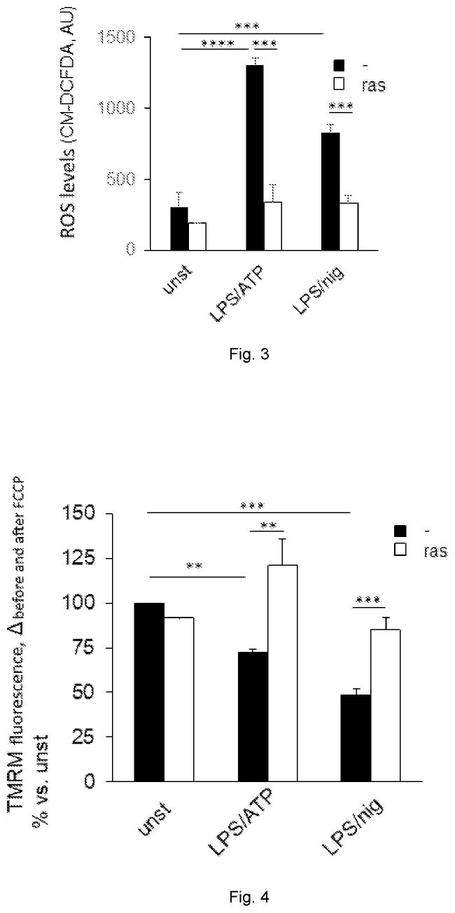

FIG. 3. MAO-B dependent H2O2 the production contributes to the production of ROS in response to inflammatory stimuli. Human monocytes isolated from the buffy coat were differentiated with GM-CSF (10 ng/ml) for 7 days and then stimulated as described in FIG. 1, in the absence or presence of rasagiline (ras, 5 μM). a) The accumulation of ROS was determined by incubating the cells with carboxymethyl dichlorofluoroscein (CM-DCFDA, 2.5 μM) after 3 h of stimulation. The images were acquired by means of fluorescence microscopy. The data are expressed as fluorescence intensity and are normalised with respect to unstimulated cells (unst). The values are the average of at least 3 independent experiments±SEM.

FIG. 4. The inhibition of MAO-B reduces the mitochondrial dysfunction induced by inflammatory stimuli. Human monocytes isolated from the buffy coat were differentiated with GM-CSF (10 ng/ml) for 7 days and then stimulated as described in FIG. 1 in the absence or presence of rasagiline (ras, 5 μM). After 8 h of stimulation, the macrophages were loaded with tetramethylrhodamine methyl ester (TMRM, 50 nM) to determine the mitochondrial membrane potential. After acquisition of images by fluorescence microscopy, the uncoupling agent FCCP (4 μM) was added to collapse the mitochondrial potential. The difference of the fluorescence intensity obtained before and after FCCP is shown in the graph and reflects the mitochondrial membrane potential. The data are expressed as fluorescence intensity and are normalised with respect to unstimulated cells (unst). The values are the average of at least 3 independent experiments±SEM.

FIG. 5. The inhibitors of MAO block the activation of the inflammasomes in human macrophages. Human monocytes isolated from the buffy coat were differentiated with GM-CSF (10 ng/ml) for 7 days and then stimulated as described in FIG. 1, in the absence or presence of pargyline (parg, 100 μM) or rasagiline (ras, 5 μM) or N-acetylcysteine (NAC, 5 mM). At the end of the stimulation, the cells were incubated with a fluorescent inhibitor of caspase-1, 660YVAD-FMK, which binds exclusively to the active form (and not to the inactive form procaspase-1). The cells were then marked with fluorescent antibodies anti-MHCII and analysed by FACS. The activity of the inflammasome was expressed as % of the cells that were positive for caspase-1 and for MHCII in relation to the total. The values are the average of at least 3 independent experiments±SEM. The graph shows that both pargyline and rasagiline significantly inhibit the activity of the inflammasome induced by two different stimuli, demonstrating the role of MAO, in particular of isoform B. The effect is similar to that observed in the presence of the antioxidant N-acetylcysteine (NAC).

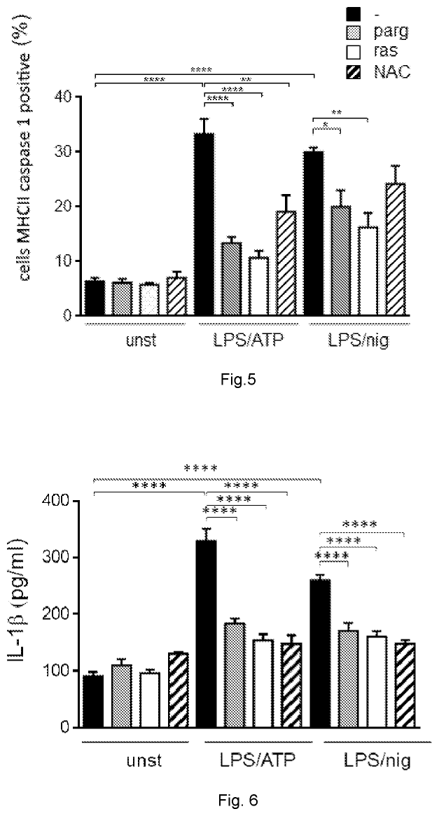

FIG. 6. The MAO inhibitors reduce the release of IL-1β in human macrophages. Human monocytes isolated from the buffy coat were differentiated with GM-CSF (10 ng/ml) for 7 days and then stimulated as described in FIG. 1, in the absence or presence of pargyline (parg, 100 μM) or rasagiline (ras, 5 μM) or N-acetylcysteine (NAC, 5 mM). The levels of IL-1β in the supernatants were quantified by means of ELISA test. The values are the average of at least 3 independent experiments±SEM. The graph shows that both pargyline and rasagiline significantly inhibit the release of IL-1β induced by two different stimuli, demonstrating the role of MAO, in particular of isoform B. The effect is similar to that observed in the presence of the antioxidant N-acetylcysteine (NAC).

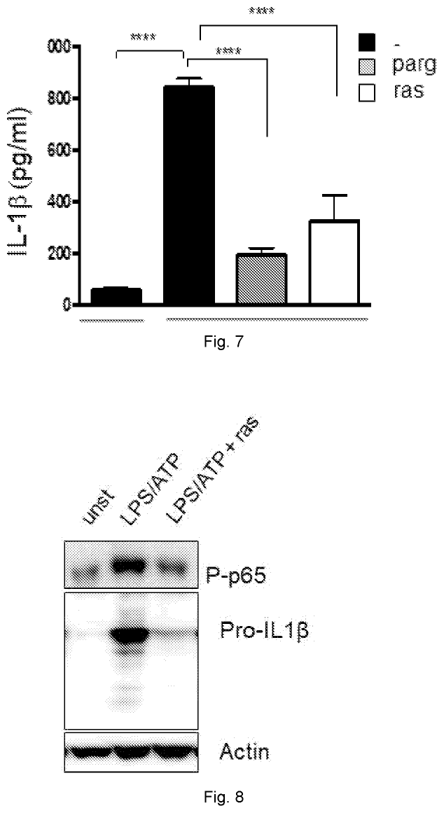

FIG. 7. The MAO inhibitors reduce the release of IL-1β in murine macrophages. Murine macrophages isolated from marrow were differentiated with GM-CSF (10 ng/ml) for 7 days and then stimulated with LPS (100 ng/ml) and ATP (5 mM, the last 30 min), for 4 h in the absence or presence of pargyline (parg, 100 μM), rasagiline (ras, 5 μM). The levels of IL-1β in the supernatants were quantified by means of ELISA test. The values are the average of at least 3 independent experiments±SEM. The graph shows that both pargyline and rasagiline significantly inhibit the release of IL-1β induced by two different stimuli, demonstrating the role of MAO, also in murine macrophages.

FIG. 8. The inhibition of MAO-B reduces the activation of NF-kB and the expression of IL-1β in murine macrophages. The values are the average of at least 3 independent experiments±SEM.

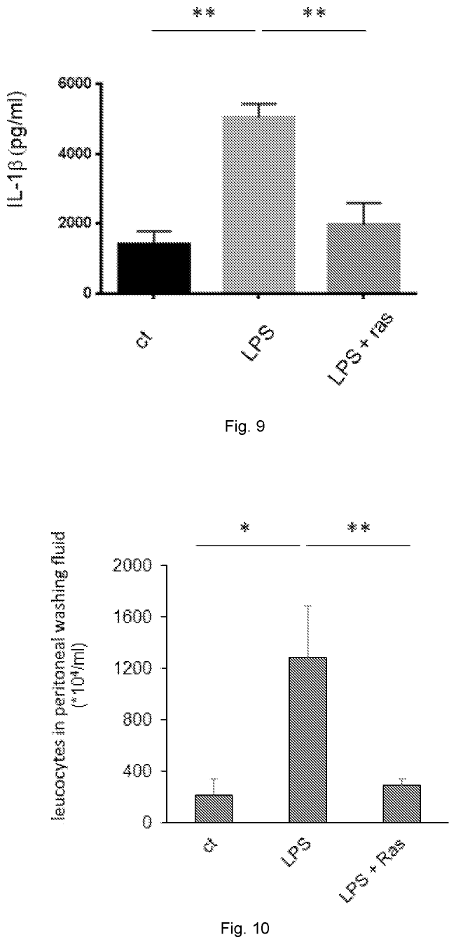

FIG. 9. The inhibition of MAO-B reduces the plasma levels of IL-1β in mice treated with LPS. Male adult mice were treated or not treated with rasagiline (ras, 0.5 mg/kg) per os and after 12 h were exposed to LPS for 8 h (10 mg/kg, ip). In parallel, a group of mice (ct) were treated with only the vehicle (PBS, ip). At the end, a sample of blood was taken and the plasma levels of IL-1β0 were quantified by means of Elisa test. The graph shows that the increase in the levels of IL-1β induced by the treatment with LPS is significantly reduced by the pretreatment with rasagiline. The values are the average of at least 3 independent experiments±SEM.

FIG. 10. The inhibition of MAO-B reduces the peritoneal exudate cell count in mice treated with LPS. Male adult mice were treated to a greater or lesser extent with rasagiline (ras, 0.5 mg/kg) per os and after 12 h were exposed to LPS for 8 h (10 mg/kg, ip). In parallel, a group of mice (ct) were treated with only the vehicle (PBS, ip). At the end, peritoneal washing with 2 ml of PBS was performed, as well as a leukocyte count. The graph shows that the increase in the number of leukocytes induced by LPS is significantly reduced by the pretreatment.

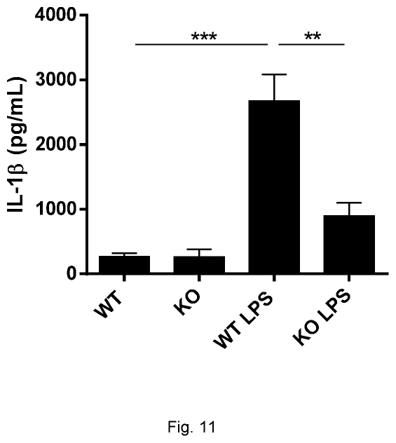

FIG. 11. The gene deletion of MAO-B reduces the plasma levels of IL-1β in mice treated with LPS. Adult male mice, both wild-type (WT) and knockout by MAOB (KO), were then treated with LPS for 8 h (10 mg/kg, ip). In parallel, a group of mice for each genotype were treated with only the vehicle (phosphate buffer saline, ip). At the end, a sample of blood was taken and the plasma levels of IL-1β were quantified by means of Elisa test. The graph shows that the increase in the levels of IL-1β induced by the treatment with LPS is significantly reduced in the knockout mice. This result constitutes the genetic validation of the role of MAOB in inflammation. The values are the average of at least 3 independent experiments±SEM.

### GLOSSARY

The term “inflammasome” in the present description has the meaning commonly used in the scientific literature. In particular, the inflammasome NLRP3, both in the literature and in the present description, is meant as a multi-protein complex that requires activation signals in order to assemble itself and generate the active form of caspase-1, which in turn converts the inactive precursors of IL-1β and IL-18 into their active forms. Inflammasome activation is a key function, mediated by the innate immune system.

The inflammasomes have been correlated with a high number of autoinflammatory and autoimmune diseases. With regard to the triggering of diseases of the inflammatory type, the inflammasomes play causal or at the least contributory roles, and exacerbate or intensify the pathology in respect of factors derived from the host.

There are different inflammasomes, which vary, for example, in the protein forming their “scaffold”. Normally, the inflammasome takes its name from the protein that forms its scaffold.

For example, the inflammasome NLRP3, the inflammasome NLRC4, the inflammasome AIM2, and others are known.

According to the present invention, throughout the description, pathologies or diseases or disorders associated with inflammasome activation means pathologies, diseases or disorders that present or manifest inflammasome activation or are even triggered, caused, wholly or partially, by such activation. MAO monoamine oxidases, group of enzymes of the oxidoreductase class, containing FAD as prosthetic group. These are flavoproteins able to oxidise various monoamines and to reduce the molecular oxygen in hydrogen peroxide. In animals they are found in plasma, kidneys, brain, muscles, and above all in the liver of mammals and are localised on the outer mitochondrial membrane.

In particular, in human beings, two isoforms of MAO called MAO-A and MAO-B have been identified. MAO-A preferably degrade serotonin, melatonin, noradrenaline, adrenaline, dopamine and tryptamine; MAO-B instead degrade dopamine, tryptamine and phenylethylamine.

The term MAO inhibitors (iMAO), or inhibitors of monoamine oxidase, is a term with a precise definition in the literature and with a meaning that is quite clear to those skilled in the art. The term, in the present description as in the literature, defines a class of substances able to reduce or block the activity of monoamine oxidases, which, as defined above, are enzymes that oxidatively metabolise the monoamines, which are compounds forming part of numerous endogenous substances, such as some neurotransmitters (such as serotonin and catecholamine adrenaline, noradrenaline, dopamine) and exogenous compounds (such as tyramine and some drugs).

The various inhibitors can be selective to a greater or lesser extent with regard to one of the two isoforms (for example the MAOA are primarily inhibited by clorgiline, whereas MAOB are selectively inhibited by selegiline and rasagiline (Youdim, M. B., Edmondson, D., and Tipton, K. F. (2006). The therapeutic potential of monoamine oxidase inhibitors. Nat Rev Neurosci 7, 295-309) or lack selectivity (as in the case of phenelzine or tranylcypromine). According to the present description, an inhibitor of monoamine oxidase type B, also referred to as iMAO, means the class of substances that is able to selectively reduce or block the activity of the monoamine oxidase type B. Thus, throughout the present description, the term inhibitor of monoamine oxidase type B can be substituted for selective inhibitor of monoamine oxidase type B or inhibitor able to selectively reduce or block the activity of monoamine oxidase type B.

Inhibitor able to “selectively reduce or block the activity of monoamine oxidase type B” means that said inhibitor does not reduce or block the activity of monoamine oxidase type A.

### DETAILED DESCRIPTION OF THE INVENTION

The present invention therefore relates to an inhibitor of monoamine oxidase type B enzyme (iMAO-B) for use in the treatment of pathologies or disorders associated with inflammasome activation.

According to the invention, pathologies or disorders associated with inflammasome activation mean all forms of the pathological type that are caused by inflammasome activation following a response of the innate immune system, or that at least present inflammasome activation following a response of the innate immune system.

For the purposes of the present description, inflammasome means any type of inflammasome, a non-limiting example of inflammasome being represented by the inflammasome NLRP3, inflammasome NLRC4 and/or the inflammasome AIM2.

In an embodiment of the invention, said pathologies or disorders are sepsis, and autoimmune and/or autoinflammatory diseases.

In accordance with an embodiment, said autoimmune and/or autoinflammatory diseases can be any autoimmune diseases known to a person skilled in the art, in particular can be any autoimmune disease apart from multiple sclerosis and type I diabetes. In an embodiment, said diseases, without limitation, can be periodic syndrome associated with cryopyrin (CAPS), periodic syndrome associated with TNF receptor 1, gout, pseudogout, irritable bowel syndrome (IBS), Crohn's disease, psoriasis, rheumatoid arthritis, systemic lupus erythematosus.

Since some diseases can be defined as being autoimmune as well as autoinflammatory, such diseases in the present description will be included in a single group for the purpose of avoiding confusion.

In an embodiment of the invention the inhibitor or the inhibitors of monoamine oxidase type B is useful in the treatment of sepsis, wherein said sepsis can be caused by any type of infection, viral or fungal bacterial.

In accordance with a further embodiment the infection treated with inhibitor or inhibitors of monoamine oxidase type B according to the invention can be any type of infection known to a person skilled in the art, for example a renal, abdominal or pulmonary infection or an infection of the circulatory stream.

Abdominal infections represent a wide variety of pathological conditions that affect all endoabdominal organs. These include the inflammation of single organs and the various forms of peritonitis (primary, secondary, tertiary), the severity of which is often dependent on the spread (isolated or diffuse). Intraperitoneal, retroperitoneal and parenchymal abscesses are also included. According to the invention the term abdominal or also intra-abdominal infections means the following pathological conditions:
infections limited to single viscera (cholecystitis, appendicitis, diverticulitis, cholangitis, pancreatitis, salpingitis, etc.), which can complicate or not into peritonitis, also in the absence of any perforation;
peritonitis, classified in turn as primary, secondary and tertiary;
intra-abdominal abscesses classified on the basis of their location and the anatomical configuration.

The term complicated intra-abdominal infections (c-IAI) is used to indicate those infections which, originating from a hollow viscus, have spread into the peritoneal space and give rise either to an abscess or to peritonitis, with distinction between the following two types:
community c-IAI: (a) moderate forms (b) severe forms;
nosocomial c-IAI: corresponding generally to post-operative infections.

Peritonitis can be classified as follows: Primary peritonitis. This is diffuse bacterial peritonitis, in the absence of perforated viscus, almost always of monomicrobial aetiology, which in turn can be subdivided into: ○spontaneous peritonitis in children; ○spontaneous peritonitis in cirrhotic adults; ○peritonitis in patients of chronic peritoneal dialysis (CAPD); ○tuberculous peritonitis and other forms of granulomatosis. Secondary peritonitis. This is localised peritonitis (often abscesses) or diffuse peritonitis originating from a defect n the continuity of the wall of the abdominal viscera. Here, a distinction can be made between 3 different groups: a) acute peritonitis caused by perforation or acute inflammation of endo-abdominal organs (peritonitis acquired in the community):—perforation and/or acute inflammation of endo-abdominal viscera;—intestinal ischaemia;—pelvic peritonitis;—bacterial translocation; b) post-operative peritonitis (nosocomial peritonitis):—caused by anastomotic dehiscence after surgery,—caused by accidental perforation and devascularisation,—caused by dehiscence of the intestinal suture line;—caused by stump dehiscence following intestinal surgery; c) post-traumatic peritonitis:—after closed abdominal trauma;—after open abdominal trauma. Tertiary peritonitis. This is constituted by delayed peritonitis-like syndromes which arise after a form of secondary peritonitis already surgically treated and is associated with a peritoneal cavity whether sterile or contaminated by microorganisms of low pathogenicity. A distinction can be made between:—peritonitis with no evidence of pathogens in the cavity;—peritonitis of fungal aetiology;—peritonitis caused by bacteria of low pathogenicity. Intra-abdominal abscesses are classified primarily on the basis of their localisation into: ○intra-peritoneal abscesses, sub-divided in turn into:—subphrenic—subhepatic—in the retrocavity of the epiploon—pelvic—paracolic—mesenteric (between the loops) ○retroperitoneal abscesses ○parenchymal abscesses—hepatic—splenic—pancreatic—renal The abscesses can be present in solitary form, multiple form, or in multiple locations.

A non-limiting example of abdominal infection according to the present description is peritonitis, cholecystitis, appendicitis, diverticulitis, cholangitis, pancreatitis, salpingitis, or intra-abdominal abscess.

According to the present description, an inhibitor of monoamine oxidase type B, also referred to as iMAO, means the class of substances that is able to selectively reduce or block the activity of the monoamine oxidase type B.

Inhibitor able to “selectively reduce or block the activity of monoamine oxidase type B” means that said inhibitor does not reduce or block the activity of monoamine oxidase type A. Any substance that has the above-mentioned activity can be used to carry out the present invention.

In accordance with an embodiment of the invention said inhibitor can be a synthetic substance or a substance of natural origin.

A non-limiting example of an inhibitor of the monoamine oxidase type B enzyme according to the present invention is represented by Lazabemide, Pargyline, Rasagiline, Selegiline, Safinamide, Catechin, Desmethoxyyangonin, Hydroxytyrosol, Klamath Algae, Piperine, Gentiana lutea or a mixture of two or more thereof.

A further subject of the invention is a pharmaceutical composition comprising one or more inhibitors of monoamine oxidase type B (iMAO) and at least one pharmaceutically acceptable carrier for use in the treatment of pathologies or disorders associated with inflammasome activation.

All of the embodiments described above for inhibitors of monoamine oxidase type B apply to the inhibitors of monoamine oxidase type B in the pharmaceutical composition according to the invention, just as the entire description of pathologies treatable with said inhibitor applies to the pathologies treatable with the composition according to the invention.

Briefly, the pathologies or disorders according to the present invention can be sepsis, and autoinflammatory and/or autoimmune diseases.

In accordance with the invention, said autoimmune and/or autoinflammatory diseases can be any autoimmune diseases known to a person skilled in the art, in particular can be any autoimmune disease apart from multiple sclerosis and type I diabetes. In an embodiment, said diseases, without limitation, can be periodic syndrome associated with cryopyrin (CAPS), periodic syndrome associated with TNF receptor 1, gout, pseudogout, irritable bowel syndrome (IBS), psoriasis, rheumatoid arthritis, Crohn's disease, systemic lupus erythematosus.

According to the invention said sepsis can be caused by any type of infection, viral or fungal bacterial, and said infection is a renal, abdominal or pulmonary infection or an infection of the circulatory stream.

According to an embodiment said abdominal infection is peritonitis, cholecystitis, appendicitis, diverticulitis, cholangitis, pancreatitis, salpingitis, or intra-abdominal abscess.

According to the present description, inhibitor of monoamine oxidase type B, also referred to as iMAO, means the class of substances that is able to selectively reduce or block the activity of monoamine oxidase type B. Inhibitor able to “selectively reduce or block the activity of monoamine oxidase type B” means that said inhibitor does not reduce or block the activity of monoamine oxidase type A. Any substance that has the above-mentioned activity can be used to carry out the present invention.

In accordance with an embodiment of the invention said inhibitor can be a synthetic substance or a substance of natural origin.

A non-limiting example of an inhibitor of the monoamine oxidase type B enzyme according to the present invention is represented by Lazabemide, Pargyline, Rasagiline, Selegiline, Safinamide, Catechin, Desmethoxyyangonin, Hydroxytyrosol, Klamath Algae, Piperine, Gentiana lutea or a mixture of two or more thereof.

The pharmaceutical composition of the invention can be formulated for an oral, systemic, or topical administration.

In accordance with an embodiment the pharmaceutical composition can therefore be formulated in the form of a tablet, pill, hard or soft gelatin, capsule, cream, emulsion, suspension, ointment, solution, powder, granules, or nanoparticles.

Since these are commercially available pharmaceutical compositions comprising inhibitors of monoamine oxidase type B as defined in the glossary and in the description, a person skilled in the art will certainly know how to provide the compositions described herein.

With regard to the embodiment in the form of nanoparticles, these can be provided in the form of a microsuspension in suitable carriers loaded with one or more inhibitors as defined in the present description by way of the techniques known to a person skilled in the art. In a particular embodiment, nanoparticles can be provided on the basis of dextran, or on the basis of beta-cyclodextrins loaded with the inhibitors of the invention. Such nanoparticles can be prepared for example as described in detail in Rodell et al, “TLR7/8-agonist-loaded nanoparticles promote the polarization of tumour-associated macrophages to enhance cancer immunotherapy” Nature Biomedical Engineering, Vol 2, August 2018, 578-588. In an embodiment, such nanoparticles can be provided in accordance with the protocol reported in the above-mentioned publication in the paragraph “Nanoparticle synthesis and characterization” in column 2 of page 585 and in column 1 of page 586 of the aforementioned article. The description provided in that paragraph is considered to be a teaching sufficient for a person skilled in the art to provide a microsuspension of nanoparticles loaded with the inhibitors of monoamine oxidase type B as defined in the glossary and in the present description. The nanoparticles according to the present invention, for example in microsuspension in a suitable carrier, can be administered orally, for example.

A further subject of the invention is a medical device comprising a pharmaceutical composition comprising one or more inhibitors of monoamine oxidase type B, iMAO, and at least one pharmaceutically acceptable carrier.

All of the embodiments described for the composition of the invention, apart from those not usable in medical devices, apply to the composition that can be comprised in the medical device as defined herein.

In an embodiment, the device can comprise an inhibitor of monoamine oxidase type B selected from Lazabemide, Pargyline, Rasagiline, Selegiline, Safinamide, Catechin, Desmethoxyyangonin, Hydroxytyrosol, Klamath Algae, Piperine, Gentiana lutea or a mixture of two or more thereof.

In an embodiment said device can be configured to release said composition (including delayed-release forms) within a predetermined time.

In accordance with a particular embodiment said device can be a medicated plaster, or a gauze.

As mentioned above, a further subject of the invention is a therapeutic method for the treatment of pathologies or disorders associated with inflammasome activation, in which an inhibitor of the monoamine oxidase type B enzyme (iMAO-B) is administered in therapeutically effective doses or a pharmaceutical composition comprising one or more inhibitors of monoamine oxidase type B (iMAO-B) and at least one pharmaceutically acceptable carrier is administered.

All of the embodiments provided above can be applied to the therapeutic method, including the application of a medical device as described above.

The examples provided below are intended to illustrate the invention and the experimentation performed by the inventors and are not intended to limit these in any way.

### EXPERIMENTS AND EXAMPLES

In order to study the role of MAO in the regulation of the activity of the inflammasome, human macrophages were isolated from the buffy coat and were stimulated by means of conventional protocols in order to activate the inflammasome in the absence or presence of rasagiline or pargyline, two iMAO having different characteristics. Pargyline is able to inhibit both the isoforms and was used as proof of principle, insofar as it was not of further clinical interest.

Rasagiline, by contrast, is selective for MAOB and is currently used to treat Parkinson's disease.

The treatment of the macrophages with LPS, LPS/ATP or LPS/nigericine (an ionophore of potassium) induces an increase in the protein levels of MAOB, as shown in FIG. 1. In addition, such treatments induce a greater availability of substrates for the enzyme. In fact, both norepinephrine and dopamine (both substrates of MAO) increase their intracellular levels, as determined by means of mass spectrometry (FIG. 2). This is consistent with a previous study that identified the phagocytes as an important source of catecholamines. The concentration of dopamine increases subsequently when its consumption is inhibited, thus blocking the MAO, suggesting that the greater availability of substrates induced by a pro-inflammatory condition contributes to the increase in the formation of products from the enzyme, in particular hydrogen peroxide.

In order to verify whether the observed greater activity of MAO induces a change in the intracellular redox homeostasis, we measured the levels of ROS. The inhibition of MAO reduces the production of ROS in response to stimulation with LPS/ATP e LPS/nigericine (FIG. 3). The excess of ROS induces mitochondrial dysfunction, as determined by measuring the mitochondrial membrane potential with the TMRM probe (FIG. 4). in order to avoid artefacts caused by the different loading capacities of the cells, at the end of each experiment an uncoupling agent (p-trifluoromethoxyphenylhydrazone (FCCP)) was added in order to collapse the mitochondrial potential. The figure shows that the pre-treatment with rasagiline is able to significantly reduce the mitochondrial depolarisation.

The authors of the invention therefore hypothesised that the overproduction of MAO-dependent H₂O₂ could be responsible for inflammasome activation. To this end, the activity of the caspase-1 was quantified by means of a specific assay. FIG. 5 shows that both pargyline and rasagiline reduce the activity of caspase-1 both induced by LPS/ATP and by LPS/nigericine. The authors then quantified the concentration of IL-1β released in the culture medium following the same pro-inflammatory stimuli. FIG. 6 shows that the levels of IL-1β produced by the catalytic activity of caspase-1 are significantly reduced in the presence of both MAO inhibitors. In addition, the data show that the degree of inhibition of the inflammasome by pargyline or rasagiline is similar to that observed by the antioxidant N-acetylcysteine (NAC). Collectively, our data suggest a crucial role of MAO, hitherto ignored, in the formation of ROS required in order to sustain inflammasome activation. Similarly, the authors of the invention observed a clear drop in the levels of IL-1β in the supernatant when murine macrophages derived from marrow (BMDM) were stimulated in vitro with LPS/ATP in the presence of inhibitors of MAO (FIG. 7). The mechanism which places the activity of the MAO in relation with inflammasome activation was therefore characterised. It is known that NF-κB is activated by LPS and induces the transcription of pro-inflammatory genes. FIG. 8 shows that rasagiline is able to reduce the activation of NF-κB and the protein levels of pro-IL-1β, suggesting a possible mechanism dependent on transcriptional control.

The authors of the invention have therefore verified the efficacy of rasagiline in the classic model of sepsis in vivo. Adult mice were treated with a single injection of LPS (10 mg/kg ip) for 8 h or of vehicle (PBS, ip). Rasagiline was administered to a group of animals per os (0.5 mg/kg) 12 h before the treatment. At the end, the animals were sacrificed and samples were taken of blood and the peritoneal exudate. FIG. 9 shows that rasagiline drastically reduces the increase of plasma levels of IL-1β induced by the treatment with LPS. In parallel, FIG. 10 shows a clear reduction of the concentration of leucocytes in the peritoneal liquid of the mice pretreated with rasagiline. Lastly, FIG. 11 shows that the mice in which MAO-B was deleted (KO) have a reduced release of IL-1β following the same treatment with LPS as compared to wild type mice (WT). On the whole, the data in vivo demonstrate a clear anti-inflammatory action of rasagiline and validate MAO-B as a pharmacological target.

1. Isolation and Differentiation of Monocytes and Macrophages, and Cell Treatment

Human monocytes derived from buffy coats obtained from healthy donors were prepared as described in Cathcart, M. K., and Bhattacharjee, A. (2014). Monoamine oxidase A (MAO-A): a signature marker of alternatively activated monocytes/macrophages. Inflammation and cell signaling 1. For macrophage differentiation, 5×10⁶ monocytes, seeded in 24-well plates, were cultivated in RPMI 20% FBS in the presence of 10 ng/ml GM-CSF (Miltenyi Biotec) for 7 days. For inflammasome activation, 2×10⁶ cells in 6-well plates were treated for 8 h with 100 ng/m1 of LPS Ultra-pure combined with ATP (1 mM) or nigericine (15 μM, added the last 30 min). The role of the MAO was tested by incubating the cells with pargyline (100 μM) or rasagiline (5 μM) for 15 min prior to stimulation.

Murine macrophages isolated from bone marrow were obtained from the tibia and femoral bone as described in Coll, R. C., Robertson, A. A., Chae, J. J., Higgins, S. C., Munoz-Planillo, R., Inserra, M. C., Vetter, I., Dungan, L. S., Monks, B. G., Stutz, A., et al. (2015). A small-molecule inhibitor of the NLRP3 inflammasome for the treatment of inflammatory diseases. Nature medicine 21, 248-255. For inflammasome activation, 10⁶ cells in 6-well plates were treated for 4 h with 100 ng/m1 of LPS

Ultra-pure and ATP (5 mM, added the last 30 min).

2. Assay of Caspase-1 Activation

The activation of caspase-1 in human macrophages was monitored by way of flow cytometry analysis. The cells, subjected to various treatments, were incubated at 37° C. for 3 h with FLICA 660-YVAD-FMK and washed according to the manufacturer's instructions (FLICA®660 Caspase-1 Assay Kit; ImmunoChemistry Technologies). The cells were then marked by fluorescent anti-MHCII antibody FITC (clone G46-6; BD Biosciences) and were analysed using a BD-FACS Calibur (Becton Dickinson), acquiring 10⁴ events. The analysis was performed using CellQuest software (Becton Dickinson) in cells positive for caspase-1 and MHCII.

3. Quantification of the Interleukin 1β

The culture media sampled from stimulated and control human and murine macrophages were stored at −80° C. The levels of IL-1β0 were determined by means of commercially available ELISA kits (eBioscience) and were developed using 3,3′,5,5′-tetramethyl benzidine (TMB). The optical densities were measured at 450 nm by means of a microplate reader (Sunrise, Tecan; Switzerland).

4. Determination of the ROS

ROS were determined by means of carboxymethyl-dichlorofluorescein (CM-DCFDA). The macrophages were plated on slides and the inflammasome was activated as described above, in the absence or presence of rasagiline (5 μM). After 3 h of stimulation the cells were loaded with CM-DCFDA (2.5 μM) for 30 min. All of the steps were performed at 37° C. with 5% CO₂. The cells were washed with PBS and the images were acquired using a Leica DMI6000B microscope (Wetzlar, Germany). The fluorescence was measured at 6-8 random intervals per slide and an average value was recorded. The fluorescence emission was performed using excitation filters of emission 488±20 nm and 645±37 nm. The data were acquired and analysed by means of Metafluor software (Universal Imaging).

5. Determination of the Mitochondrial Membrane Potential

The mitochondrial membrane potential was measured on the basis of the accumulation of tetramethylrhodamine methyl ester (TMRM, Molecular Probes) as described in Sorato, E., Menazza, S., Zulian, A., Sabatelli, P., Gualandi, F., Merlini, L., Bonaldo, P., Canton, M., Bernardi, P., and Di Lisa, F. (2014). Monoamine oxidase inhibition prevents mitochondrial dysfunction and apoptosis in myoblasts from patients with collagen VI myopathies. Free radical biology & medicine 75, 40-47. The myotubes of patients with DMD and healthy donors were obtained as described above and treated with H₂O₂ (100 μM) for 30 minutes in the absence or presence of pre-treatment with ZP049 (1 μM, added 20 minutes before the hydrogen peroxide). The medium was then substituted with a medium devoid of serum integrated with TMM 25 nM for 30 minutes, and the cell fluorescence images were acquired using a Leica microscope (Wetzlar, Germany) DMI6000B. The data were acquired and analysed by means of Metafluor software (Universal Imaging). An excitation of 540±20 nm and a 590 nm longpass emission filter were used to reveal the fluorescence. The dusters of various mitochondria were identified as regions of interest (ROI) and the areas not containing cells were considered as background. To exclude artefacts caused by the various loading capacities of the various cells, which could be interpreted erroneously as differences Δψm, sequential digital images were acquired before and after the addition of carbonyl cyanide-p-trifluoromethoxyphenylhydrazone (FCCP, 4 μM), a protonophore which completely depolarises mitochondria. The Δψm was calculated as the difference in the fluorescence intensity of the TMRM before and after FCCP of ROI from at least 30 cells. Experiments with various agents described above were always performed in relation to untreated cells.

6. Treatment in Vivo

Male mice (strain C57BL/6J) of age 8-10 weeks and weight approximately 30 gr. were subjected to an intraperitoneal injection with LPS (10 mg/kg, L4130, Sigma-Aldrich) or vehicle (saline solution, called PBS). One group of mice was treated per os with the inhibitor of MAOB rasagiline (0.5 mg/kg, Sigma Aldrich). The treated animals were sacrificed 8 hours after the injection (considered as time 0), by means of cervical dislocation. Immediately after the sacrifice an intraperitoneal washing was performed with 2 ml of PBS and any exudate present was collected to determine the leukocyte count. In addition, samples of blood were taken and frozen after obtaining the plasma for analysis of IL-1β by means of ELISA test.

7. Analysis of the Data and Statistical Procedures

The data are expressed as the average±SEM. The comparison between the two groups was performed by means of the Student t test and values with p<0.05 were considered significant.

1. A method for treating pathologies or disorders associated with inflammasome activation, wherein said pathologies or disorders are autoinflammatory and/or autoimmune diseases, wherein said method comprises:
administering an inhibitor of monoamine oxidase type B enzyme iMAO-B to a subject in need thereof, wherein said inhibitor is Rasagiline, Safinamide, or a mixture thereof.

2. The method according to claim 1, wherein said autoinflammatory and/or autoimmune diseases are periodic syndrome associated with cryopyrin (CAPS), periodic syndrome associated with TNF receptor 1, gout, pseudogout, irritable bowel syndrome, psoriasis, rheumatoid arthritis, systemic lupus erythematosus.

3-6. (canceled)

7. The method according to claim 1, wherein said treatment of pathologies or disorders associated with inflammasome activation comprises administering said inhibitor in a pharmaceutical composition comprising at least one pharmaceutically acceptable carrier.

8-12. (canceled)

13. The method according to claim 7, wherein said pharmaceutical composition is formulated for an oral, systemic, or topical administration.

14. The method according to claim 7, wherein said pharmaceutical composition is in the form of a tablet, pill, hard or soft gelatin, capsule, cream, emulsion, suspension, ointment, solution, powder, granules, or nanoparticles.

15. The method according to claim 1, wherein said treatment of pathologies or disorders associated with inflammasome activation comprises administering said inhibitor through a medical device.

16. (canceled)

17. The method according to claim 15, wherein said device is configured for releasing said inhibitor in a predetermined time.

18. The method according to claim 15, wherein said device is a medicated plaster, or a gauze.
